(19) United States
(12) Patent Application Publication
(10) Pub. No.: US 2015/0018746 A1
(43) Pub. Date: Jan. 15, 2015

(54) DEVICE FOR USE IN GLAUCOMA SURGERY

(71) Applicant: GEUDER AG, Heidelberg (DE)

(72) Inventor: Lars-Olof Hattenbach, Ludwigshafen (DE)

(21) Appl. No.: 14/371,381

(22) PCT Filed: Jan. 9, 2013
(86) PCT No.: PCT/DE2013/200001

§ 371 Date: Jul. 9, 2014

(30) Foreign Application Priority Data
Jan. 12, 2012 (DE) 10 2012 200 411.4

(51) Int. Cl. A61F 9/007

(52) U.S. Cl. CPC A61F 9/00781
USPC 604/8

(57) ABSTRACT

The invention relates to a device for use in glaucoma surgery intended for implantation in the eye. The device comprises a flat head region (8), a probe (6) which adjoins the head region (8), and a relief channel (7) which extends through the head region (8) and the probe (6) and is open at both sides. The design and dimensions of the head region (8) and of the probe (6) are such that the probe (6) can be inserted by its free end through the sclera (4) into the posterior chamber (10) of the eye in order to reduce an excessive intraocular pressure when the conjunctiva (3) is open, and such that, in the inserted position, the lower side of the head region (8) comes into abutment with the sclera (4), and the upper side of the head region (8) can be covered by closing the conjunctiva (3) such that drainage can be performed from the posterior chamber (10) into a gap (11) between the conjunctiva (3) and the sclera (4).

### CROSS REFERENCE TO RELATED APPLICATIONS

This application is a national stage application, filed under 35 U.S.C. §371, of International Application No. PCT/DE2013/200001, filed Jan. 9, 2013, which claims priority to German Application No. 10 2012 200 411.4, filed Jan. 12, 2012, both of which are hereby incorporated by reference in their entirety.

### BACKGROUND

1. Technical Field

The invention relates to a device for use in glaucoma surgery, in particular, for implantation in the eye. Furthermore, the invention relates to the use of such a device in glaucoma surgery that is intended for implantation in the eye as well as a method for using a corresponding device in glaucoma surgery that is intended for implantation in the eye, for the purpose of lowering the abnormal elevation of the intraocular pressure.

The device is intended for use in glaucoma surgery, in particular, for the surgical treatment of glaucoma. The term “glaucoma” includes a group of ocular diseases of varying etiology that can lead to the loss of the optic nerve fibers. The result of such a disease is the characteristic loss of the field of vision and in the extreme case blindness of the eye. An abnormal elevation of the intraocular pressure is considered to be the most important risk factor.

2. Description of Related Art

Many people suffer from an abnormal elevation of the intraocular pressure. Correspondingly many people suffer from glaucoma, in Germany alone, about one million people.

In addition to a pharmacological treatment of glaucoma, a number of different surgical procedures are available to treat glaucoma. In this case the preferred method of treatment is, depending on the specific type of disease, the drainage of the aqueous humor fluid, as a result of which it is possible to lower the intraocular pressure. Trabeculectomy (TE) is deemed to be, besides other types of filtering surgical intervention, the standard technique that is used in penetrating glaucoma surgery. First, an artificial hole is made in the cornea (sclera). Then this hole is loosely closed and covered with conjunctiva. Different shunt systems are used to drain the aqueous humor fluid.

Based on the principle of trabeculectomy, the subconjunctival drainage is known, according to which a channel system is formed between the anterior chamber of the eye and a scleral pocket or more specifically a subconjunctival filtering bleb. In order to achieve suitable drainage, the Ahmed valve is known from practical use.

Another surgical approach is the trabecular meshwork bypass, according to which the shunt obtains an open channel in the trabecular meshwork through the implantation of a micro tube. The iStant [sic] is known from practical use for this purpose.

An additional method uses a drainage system into the suprachoroidal space. The SOLX shunt is known from practical use for this purpose.

All of the glaucoma shunts, which are known from practical use, are based on draining the fluid from the anterior chamber of the eye.

The devices, which are known from practical use for lowering the abnormal elevation of the intraocular pressure, are difficult to use, in particular during the implantation. In addition, there is also the risk of misplacement and/or “wandering.”

### BRIEF SUMMARY

Therefore, the object of the present invention is to provide a device that is intended for use in glaucoma surgery, for implantation in the eye and that is as easy as possible to use and at the same time is safe and can be implanted for the purpose of lowering the abnormal elevation of the intraocular pressure. Another object of the present invention is to guarantee a secure seat in the implanted state, i.e. in the eye. Finally the device shall be inexpensive to manufacture.

The above engineering object is achieved by means of the features disclosed in patent claim 1. According to claim 1, the inventive device for use in glaucoma surgery, in particular, for implantation in the eye, comprises a flat head region, a probe that adjoins the head region, and a relief channel, which extends through the head region and the probe and is open on both sides.

The head region is an extremely flat region, which is constructed like a flat nail head or the head of a thumbtack. As a result, the head region forms a bearing surface with its bottom side and, therefore, makes it possible to position the probe with very high precision.

The relief channel is formed by a tube that is open on both sides. In this case the tube ends in the head region in such a way that the relief channel extends through the head region and opens outwards. As a result, the relief channel is open at both ends.

The head region and the probe are dimensioned and configured in such a way that when the conjunctiva is open, the probe, which is designed for the purpose of lowering the abnormal elevation of the intraocular pressure, can be inserted with the free end through the cornea or more specifically the sclera as far as into the posterior chamber of the eye. The result is that the size or rather the length of the probe is defined, so that in the inserted position the head region comes to rest against the sclera with its bottom side.

Working on the aforesaid, the device according to the invention is based on the idea of draining, not in accordance with the common practice known from the prior art, the fluid from the anterior chamber of the eye. On the contrary, the object of the invention is to place the glaucoma shunt where the abnormal elevation of the intraocular pressure acts, in particular, in the posterior chamber of the eye. Correspondingly the device with the probe is dimensioned in such a way that it can be inserted through the sclera as far as into the posterior chamber of the eye, so that a flow communication between the posterior chamber of the eye is established outwards. After the insertion of the device, the special function of the head region is important, because the conjunctiva is closed again by means of the head region and is routinely sutured. Consequently the originally opened conjunctival flap covers the upper side of the head region, so that drainage from the posterior chamber into a space between the conjunctiva and the sclera is achieved.

At this point it is explicitly to be noted that the device according to the invention is a kind of trocar tube, which is used for purposes of an implant designed for drainage from the posterior chamber into the region between the conjunctiva and the sclera.

In order not to provoke a detachment of the retina, the pars plana region lends itself to the implantation. After the preparation or rather the opening of the conjunctiva, it is possible to place with a minimum of effort one or more shunts in the form of the device according to the invention, i.e. in the form of very small and flat trocars, under the conjunctiva. After the placement of the trocars, the conjunctival flap, which is created in the direction of the opening, can be closed again by means of the trocar head(s) and sutured, so that the device according to the invention can be used for drainage into the space between the conjunctiva and the sclera.

The head region is concavely curved in an advantageous way at least on the bottom side, i.e. on the side facing the probe, in conformity with the geometry of the eye. As a result, a secure seat of the device directly at or on the sclera is possible. It is also advantageous, if the head region is configured so as to be at least slightly convex on the upper side, i.e. on the side facing away from the probe. This feature facilitates the covering of the head region with the conjunctiva or more specifically the conjunctival flap.

It is also conceivable that the upper side of the head region comprises the drainage facilitating, preferably radial ribs, grooves or the like, in order to guide the fluid emerging from the relief channel, preferably with the use of capillary forces, into the space between the sclera and the conjunctiva. Once more this arrangement promotes the drainage action.

The device is made in an advantageous manner of a biocompatible material; at least the device is coated with a biocompatible material. In accordance with a specific embodiment the device can be made of ceramic, glass ceramic or glass. The material can be made permeable or semi-permeable, as a result of which the drainage action is facilitated once again.

As already stated above, the device according to the invention is intended for use in glaucoma surgery; or more specifically it is used in glaucoma surgery for implantation in the eye for the purpose of lowering the abnormal elevation of the intraocular pressure. Correspondingly a method for using a device according to the invention, i.e. in glaucoma surgery for the implantation in the eye, and in particular, for the purpose of lowering the abnormal elevation of the intraocular pressure, is also claimed. In this respect it is essential that when the conjunctiva is open, the probe be inserted with the free end through the sclera as far as into the posterior chamber of the eye. At the same time at the head region, which rests against the sclera, said head region is covered by closing the conjunctiva or rather by closing the conjunctival flap that was created while opening the conjunctiva, so that drainage from the posterior chamber into the space between the conjunctiva and the sclera is achieved.

### BRIEF DESCRIPTION OF THE FIGURE

At this point there are a number of possibilities for designing and further developing the teaching of the present invention in an advantageous way. To this end, reference is made, on the one hand, to the claims subordinated to the patent claim 1 and, on the other hand, to the following description of a preferred embodiment of the invention with reference to the drawings. In conjunction with the description of the preferred embodiment of the invention with reference to the drawings, generally preferred embodiments and further developments of the teaching are also described. The drawings show in

FIG. 1 in schematic form a view of a part of the human eye in the pars plana region with the conjunctiva open and the inventive device already inserted; and

FIG. 2 in schematic form a view of a part of the human eye from FIG. 1, where the conjunctiva has already been closed again over and beyond the trocar head by means of the conjunctival flap, which had been created when said conjunctiva was opened.

### DETAILED DESCRIPTION OF VARIOUS EMBODIMENTS

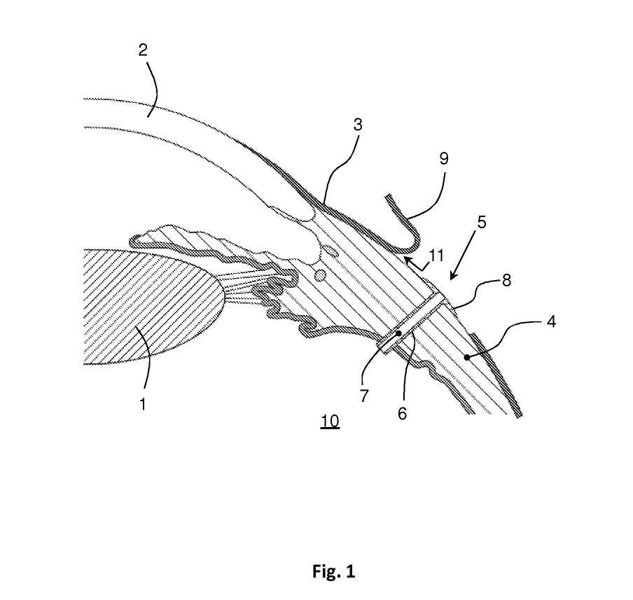

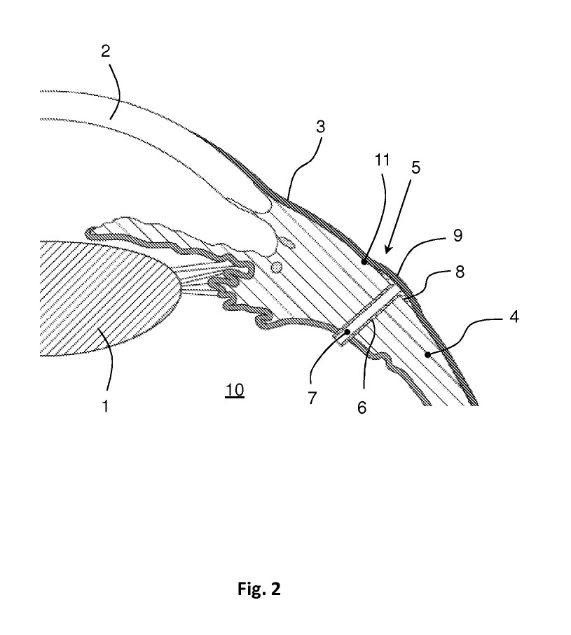

FIGS. 1 and 2 show in schematic form a sectional view of a part of the human eye in a highly simplified representation, where the lens 1, the cornea 2, the conjunctiva 3 and the sclera 4 are marked as the essential components.

In the drawing from FIG. 1, the inventive device 5 is inserted for implant purposes while the conjunctiva 3 is open in such a way that the probe 6 extends completely through the sclera 4. The relief channel 7, which is open on both sides, extends through the probe 6.

The insertion of the device 5 or more specifically the probe 6 is defined by the head region 8. To put it more succinctly, the head region 8 sits with its bottom side close to the sclera 4.

According to the drawing in FIG. 2, the flap 9, which is made in order to open the conjunctiva 3, is placed over the head region 8 of the device 5; and the conjunctiva 3 is sutured. In this way the device 5 is implanted in its entirety, with the result that a drainage system is created between the posterior chamber 10 and a space 11 between the sclera 4 and the conjunctiva 3. Finally a pressure relief from the posterior chamber 10 is provided by using the device according to the invention.

The head region is an extremely flat region, which is constructed like a flat nail head or the head of a thumbtack. As a result, the head region forms a bearing surface with its bottom side and, therefore, makes it possible to position the probe with very high precision.

The relief channel is formed by a tube that is open on both sides. In this case the tube ends in the head region in such a way that the relief channel extends through the head region and opens outwards. As a result, the relief channel is open at both ends.

The head region and the probe are dimensioned and configured in such a way that when the conjunctiva is open, the probe, which is designed for the purpose of lowering the abnormal elevation of the intraocular pressure, can be inserted with the free end through the cornea or more specifically the sclera as far as into the posterior chamber of the eye. The result is that the size or rather the length of the probe is defined, so that in the inserted position the head region comes to rest against the sclera with its bottom side.

Working on the aforesaid, the device according to the invention is based on the idea of draining, not in accordance with the common practice known from the prior art, the fluid from the anterior chamber of the eye. On the contrary, the object of the invention is to place the glaucoma shunt where the abnormal elevation of the intraocular pressure acts, in particular, in the posterior chamber of the eye. Correspondingly the device with the probe is dimensioned in such a way that it can be inserted through the sclera as far as into the posterior chamber of the eye, so that a flow communication between the posterior chamber of the eye is established outwards. After the insertion of the device, the special function of the head region is important, because the conjunctiva is closed again by means of the head region and is routinely sutured. Consequently the originally opened conjunctival flap covers the upper side of the head region, so that drainage from the posterior chamber into a space between the conjunctiva and the sclera is achieved.

At this point it is explicitly to be noted that the device according to the invention is a kind of trocar tube, which is used for purposes of an implant designed for drainage from the posterior chamber into the region between the conjunctiva and the sclera.

In order not to provoke a detachment of the retina, the pars plana region lends itself to the implantation. After the preparation or rather the opening of the conjunctiva, it is possible to place with a minimum of effort one or more shunts in the form of the device according to the invention, i.e. in the form of very small and flat trocars, under the conjunctiva. After the placement of the trocars, the conjunctival flap, which is created in the direction of the opening, can be closed again by means of the trocar head(s) and sutured, so that the device according to the invention can be used for drainage into the space between the conjunctiva and the sclera.

The head region is concavely curved in an advantageous way at least on the bottom side, i.e. on the side facing the probe, in conformity with the geometry of the eye. As a result, a secure seat of the device directly at or on the sclera is possible. It is also advantageous, if the head region is configured so as to be at least slightly convex on the upper side, i.e. on the side facing away from the probe. This feature facilitates the covering of the head region with the conjunctiva or more specifically the conjunctival flap.

It is also conceivable that the upper side of the head region comprises the drainage facilitating, preferably radial ribs, grooves or the like, in order to guide the fluid emerging from the relief channel, preferably with the use of capillary forces, into the space between the sclera and the conjunctiva. Once more this arrangement promotes the drainage action.

The device is made in an advantageous manner of a biocompatible material; at least the device is coated with a biocompatible material. In accordance with a specific embodiment the device can be made of ceramic, glass ceramic or glass. The material can be made permeable or semi-permeable, as a result of which the drainage action is facilitated once again.

As already stated above, the device according to the invention is intended for use in glaucoma surgery; or more specifically it is used in glaucoma surgery for implantation in the eye for the purpose of lowering the abnormal elevation of the intraocular pressure. Correspondingly a method for using a device according to the invention, i.e. in glaucoma surgery for the implantation in the eye, and in particular, for the purpose of lowering the abnormal elevation of the intraocular pressure, is also claimed. In this respect it is essential that when the conjunctiva is open, the probe be inserted with the free end through the sclera as far as into the posterior chamber of the eye. At the same time at the head region, which rests against the sclera, said head region is covered by closing the conjunctiva or rather by closing the conjunctival flap that was created while opening the conjunctiva, so that drainage from the posterior chamber into the space between the conjunctiva and the sclera is achieved.

Finally it is explicitly noted that the above described exemplary embodiment of the device according to the invention serves only to explain the claimed teaching, but the claimed teaching is not restricted to the exemplary embodiment.

### LIST OF REFERENCE NUMERALS

2 cornea
3 conjunctiva
4 sclera, cornea
5 device (comprising the probe 6, the relief channel 7 and the head region 8)
6 probe
7 relief channel
8 head region
9 flap (the conjunctiva)
10 posterior chamber (of the eye)
11 space (between the sclera and the conjunctiva)

1-9. (canceled)

10. Device for use in glaucoma surgery, in particular, for implantation in the eye, said device comprising:
a flat head region (8);
a probe (6) that adjoins the head region (8); and
a relief channel (7), which extends through the head region (8) and the probe (6) and is open on both sides,
wherein:
the head region (8) and the probe (6) are dimensioned and configured in such a way that when the conjunctiva (3) is open, the probe (6), which is designed for the purpose of lowering the abnormal elevation of the intraocular pressure, can be inserted with the free end through the sclera (4) as far as into the posterior chamber (10) of the eye;
in the inserted position the head region (8) comes to rest against the sclera (4) with its bottom side; and
the upper side of the head region (8) can be covered by closing the conjunctiva (3), so that drainage from the posterior chamber (10) into a space (11) between the conjunctiva (3) and the sclera (4) can be achieved.

11. Device, as claimed in claim 10, wherein the head region (8) is configured so as to be concavely curved at least on the bottom side, the bottom side being on the side facing the probe (6), in conformity with the geometry of the eye.

12. Device, as claimed in claim 10, wherein the head region (8) is configured so as to be at least slightly convex on the upper side, the upper side being the side facing away from the probe (6).

13. Device, as claimed in claim 10, wherein the upper side of the head region (8) comprises one or more drainage facilitating elements.

14. Device, as claimed in claim 13, wherein the one or more drainage facilitating elements comprise at least one of radial ribs or groove.

15. Device, as claimed in claim 10, wherein the device is at least one of coated with or made of a biocompatible material.

16. Device, as claimed in claim 10, wherein the device is made of at least one of ceramic, glass ceramic, or glass.

17. Device, as claimed in claim 15, wherein the material is designed so as to be at least one of permeable or semi-permeable.

18. Use of a device, as claimed in claim 10, in glaucoma surgery for implantation in the eye for the purpose of lowering the abnormal elevation of the intraocular pressure, wherein when the conjunctiva (3) is open, the probe (6) is inserted with the free end through the sclera (4) as far as into the posterior chamber (10) of the eye; and wherein at the head region (8), which rests against the sclera (4), said head region is covered by closing the conjunctiva (3), so that drainage from the posterior chamber (10) into a space between the conjunctiva (3) and the sclera (4) is achieved.

19. Method for using a device, as claimed claim 10, in glaucoma surgery for implantation in the eye for the purpose of lowering the abnormal elevation of the intraocular pressure, wherein when the conjunctiva (3) is open, the probe (6) is inserted with the free end through the sclera (4) as far as into the posterior chamber (10) of the eye; and wherein at the head region (8), which rests against the sclera (4), said head region is covered by closing the conjunctiva (3), so that drainage from the posterior chamber (10) into a space (11) between the conjunctiva (3) and the sclera (4) is achieved.
